(19) United States
(12) Patent Application Publication
(10) Pub. No.: US 2024/0032528 A1
(43) Pub. Date: Feb. 1, 2024

(54) A SYSTEM FOR STORING OR CULTIVATING OF AN ORGAN OR TISSUE MODEL AND USES THEREOF

(71) Applicant: Polbionica Sp. z o. o., Warszawa (PL)

(72) Inventors: Michal WSZOLA, Warszawa (PL); Marta KLAK, Pila (PL); Andrzej BERMAN, Warszawa (PL); Tomasz DOBRZANSKI, Warszawa (PL); Igor ZAMORA, Warszawa (PL); Dawid KUBINKIEWICZ, Warszawa (PL); Mateusz SZCZYGIELSKI, Stara Wies (PL)

(73) Assignee: Polbionica Sp. z o. o., Warszawa (PL)

(21) Appl. No.: 18/268,481

(22) PCT Filed: Dec. 22, 2021
(86) PCT No.: PCT/IB2021/062190

§ 371 Date: Jun. 20, 2023

(30) Foreign Application Priority Data
Dec. 22, 2020 (EP) 20461600.7

(51) Int. Cl. A01N 1/02

(52) U.S. Cl. CPC A01N 1/021; A01N 1/0247; A01N 1/0252; A01N 1/0284

(57) ABSTRACT

The invention relates to a system for storing or cultivating of an organ or a tissue model, and to a use thereof. The system is designed for verifying the functioning of a harvested organ or a tissue model and allows for treating it. The system may be used to cultivate organs or tissue models harvested from donors and/or produced using other techniques, such as using 3D and/or 4D bioprinting or electrospinning technology, and comprises: a chamber (2) for the organ or tissue model equipped with means for measuring and adjusting temperature of perfusion fluid in the chamber, a perfusion fluid container (1) equipped with means for measuring and adjusting temperature of the perfusion fluid therein; means (4) for measuring glucose concentration in the perfusion fluid and for dosing glucose into the perfusion fluid; means (3) for measuring pH of the perfusion fluid and for dosing substance for adjusting the pH of the perfusion fluid; means for contactless mixing of the perfusion fluid; and means for measuring and controlling of flow parameters of the perfusion fluid.

The invention relates to a system for storing or cultivating of an organ or tissue model having a flow or vascular system and to a use thereof. The system is designed for, including without limitation, verifying the functioning of a harvested organ or a tissue model and allows for treating it. The system may be used to cultivate organs or tissue models harvested from donors and/or produced using other techniques, such as using 3D and/or 4D bioprinting or electrospinning technology. The system may be used for the flow culture of organs that cannot be harvested or treated otherwise in a patient. The term “organ flow system” relates to artificially produced organs and denotes a system with formed channels. The term “organ vascular system” denotes a system of vessels with endothelial cells, either harvested from donors or produced artificially. The term “storing” relates to keeping an organ or a tissue model at a low temperature at reduced metabolism or prior to colonisation with cells. The term “cultivating” relates to keeping an organ or a tissue model colonised with cells in a normothermic environment corresponding to in vivo conditions. The term “tissue model” denotes a three-dimensional structure containing living cells suspended in a biomaterial, which may additionally contain a vascular system.

U.S. Pat. No. 9,756,851B2 discloses a composition, a method and a device for keeping a harvested organ viable before implantation. The organ perfusion device comprises a preservation chamber for storing the organ. A perfusion circuit is provided with the first line for supplying oxygenated fluid to the organ and a second line for discharging used fluid from the organ. The perfusion device also comprises a device functionally connected to the perfusion circuit for keeping the organ at a generally normothermic temperature. In addition, the device contains means for controlling pressure of the perfusion fluid, oxygenation means for oxygenating at least some of said fluid, filtration means and flow control means for controlling the flow of at least some of said fluid.

Patent document WO1996029865A1 describes an organ perfusion apparatus capable of perfusing organs at temperatures close to normal, using blood or other oxygen-carrying substances. The apparatus allows to assess the viability of organs using on-line measurements of physiological efficiency. Embodiments use a computer-controlled blood pump in order to characterise physiological conditions of perfusion. The apparatus comprises parts that allow for replenishing lost circulating volume and for infusing nutrients, medicaments and components of perfusion fluid in order to help to maintain or regenerate organs. The apparatus can also adjust pressure, pH and temperature, automatically measure rate of production of urine, bile, pancreatic duct secretions or other physiological exudates, and determine flowrate of blood or perfusate, vascular resistance and organ swelling.

US2017339945A1 discloses an apparatus for multi-organ perfusion, selected from a group comprising heart, liver, kidneys and lungs, comprising a base unit, configured to be detachably connected to a perfusion module for organ perfusion. The base unit is provided with tubes to connect a perfusate source to the organ so that perfusate circulates through the organ, first and second pumps, connected to the tubes for driving circulation of the perfusate in the tubes and a controller, configured and connected to control the first and second pumps in order to control the circulation of the perfusate through the organ. The controller can control the first and second pumps for the purposes of organ perfusion depending on perfusion parameters selected, based on the type of organ.

Document EP1879997A2 discloses a portable organ perfusion device with an organ chamber for supporting an organ immersed in perfusion fluid. The pump circulates the perfusion fluid around a circuit containing a pump, a heat exchanger for cooling the perfusion fluid, an oxygenator to oxygenate the perfusion fluid and a device for ensuring a constant supply of fluid to the organ vascular system. A by-pass channel provides a fluid connection, allowing excess fluid to bypass the organ. The device further comprises an oxygen source, sensors, a power supply and a control unit for receiving information from sensors and providing control instructions to the control means.

Solutions are being sought that would ensure efficient control of maintaining normothermic conditions, regulation of fluids or pH control for an efficient, more automatic and accurate repair or cultivating of organs and tissue models. The objective of the invention is to provide suitable conditions for cultivating and storing organs harvested from deceased donors, such as pancreas, liver, kidney, lung, heart, small intestine, large intestine, thyroid, skin, brain, or produced using other techniques, e.g. using 3D and/or 4D bioprinting technology such as tissue models printed with a vascular system. The term “bionic organs” relates to both tissue models printed in 3D and/or 4D technology directly with cells, and scaffolds alone (vascular systems and tissue models), which are only subsequently colonised with respective cells. The term “3D bioprinting” relates to a formation of three-dimensional structures containing viable cells using additive manufacturing technologies (layer-by-layer material application). In 4D bioprinting, fourth dimension is time, and the technique yields bands of materials that are formed to later transform into a previously defined object (shape).

In a first aspect of this invention, a system is provided for storing or cultivating an organ or a tissue model, preferably harvested from a donor, preferably selected from pancreas, liver, kidney, lung, heart, small intestine, large intestine, thyroid, skin, brain, or produced using other techniques, preferably using 3D bioprinting and/or 4D bioprinting and/or electrospinning technologies, preferably in a form of organs and/or tissue models printed with flow or vascular systems, comprising:
a chamber for an organ or a tissue model equipped with means for measuring and adjusting temperature of the perfusion fluid placed in the chamber during the operation of the system, wherein said means for measuring and adjusting temperature of the perfusion fluid preferably comprise a water jacket
a perfusion fluid container, equipped with means for measuring and adjusting temperature of the perfusion fluid in the container during operation of the system, preferably comprising a heating-cooling plate, wherein the perfusion fluid container is connected to the organ chamber by at least one first line through which the perfusion fluid can flow from the perfusion fluid container to the organ chamber during operation, and at least one second line through which the perfusion fluid can flow from the organ chamber to the perfusion fluid container during operation
means for measuring glucose concentration in the perfusion fluid and for dosing glucose into the perfusion fluid
means for measuring pH of the perfusion fluid and for dosing substance for adjusting the pH of the perfusion fluid
means for contactless mixing of the perfusion fluid
means for measuring and controlling of flow parameters of perfusion fluid, including perfusion fluid pressure and/or perfusion fluid output, expressed as a unit of volume per unit of time
optionally, means for measuring and controlling one or more parameters, selected from: lactate concentration in the perfusion fluid, concentration of sodium ions in the perfusion fluid, concentration of chlorine ions in the perfusion fluid, concentration of potassium ions in the perfusion fluid, oxygenation level of the perfusion fluid, wherein during operation of the system the system is configured to circulate said perfusion fluid between the organ chamber and the perfusion fluid container.

The perfusion fluid flow output denotes the perfusion fluid flow rate, expressed in a unit of volume per unit of time. The term “organ chamber” denotes a chamber suitable for storing or cultivating of an organ or tissue model.

Preferably, the system for storing or cultivating an organ or tissue model comprises system control means, configured and programmed to obtain measurement values for at least one parameter selected from: temperature of the perfusion fluid in the organ chamber, temperature of the perfusion fluid in the perfusion fluid container, concentration of glucose in the perfusion fluid, pH of the perfusion fluid, parameters of perfusion fluid flow, including pressure of the perfusion fluid and/or output of the perfusion fluid, expressed as a unit of volume per unit of time, concentration of lactates in the perfusion fluid, concentration of sodium ions in the perfusion fluid, concentration of chlorine ions in the perfusion fluid and concentration of potassium ions in the perfusion fluid, oxygenation, wherein the system control means are further configured and programmed for automatic and/or manual adjustment based on predetermined criteria of at least one parameter selected from: temperature of the perfusion fluid in the organ chamber, temperature of the perfusion fluid in the perfusion fluid container, concentration of glucose in the perfusion fluid, pH of the perfusion fluid, parameters of perfusion fluid flow, including pressure of the perfusion fluid and/or output of the perfusion fluid, lactate concentration in the perfusion fluid, sodium ion concentration in the perfusion fluid, chlorine ion concentration in the perfusion fluid and potassium ion concentration in the perfusion fluid, oxygenation.

Preferably, the system for storing or cultivating an organ or tissue model further comprises means for measuring and controlling the oxygenation of perfusion fluid and/or means for air removal form the perfusion fluid, wherein the system control means are preferably configured and programmed to obtain perfusion fluid oxygenation measurement values and automatic and/or manual adjustment of perfusion fluid oxygenation based on predetermined criteria.

Preferably, the system for storing or cultivating an organ or tissue model further comprises means for measuring of loss and replenishment of the perfusion fluid, preferably based on a reading of weight of the perfusion fluid removed from the system, wherein the system control means are preferably configured and programmed for automatic and/or manual replenishment of the perfusion fluid based on predetermined criteria.

Preferably, the means for contactless mixing of the perfusion fluid are provided as a swinging mechanism for swinging of the perfusion fluid container, wherein the system control means are preferably configured and programmed to automatically and/or manually adjust the swinging of the perfusion fluid container.

Preferably, oxygenation of the perfusion fluid is performed with an oxygenator, which is peripherally connected to the perfusion fluid container to form an oxygenator circuit, and the means for mixing the perfusion fluid are provided in a form of a cylindrical design of the perfusion fluid container, in which, during operation of the system, the perfusion fluid is mixed by placing the inlet and outlet of the oxygenator circuit on opposite sides of the perfusion fluid container, tangentially to the cylindrical walls of the perfusion fluid container.

Preferably, the system comprises means for taking samples of the perfusion fluid.

Preferably, the organ chamber comprises a rotating mechanism which allows the organ chamber to rotate during operation of the system, wherein the system control means are preferably configured and programmed to automatically and/or manually adjust the rotation of the organ chamber. Preferably, the system control means are configured and programmed to rotate the organ chamber by 180° every 30 minutes for at least two hours or at least four times by 90° every 15-60 minutes for at least two hours.

Preferably, the organ chamber and/or the perfusion fluid container and/or the means for measuring and controlling the oxygenation of the perfusion fluid has/have means for adjusting temperature in the range from 0 to 37° C.

Preferably, the system further comprises additional means for dosing medicaments and/or nutrients and/or perfusion fluid components, wherein the system control means are preferably configured and programmed for automatic and/or manual dosing of medicaments and/or nutrients and/or perfusion fluid components based on predetermined criteria.

Preferably, air is removed from the perfusion fluid by means of an air bubble eliminator mounted on the first line between the perfusion fluid container and the organ chamber, and the movement of the perfusion fluid between the perfusion fluid container and the organ chamber and the other way round during operation of the system is performed by means of at least one pump, preferably a peristaltic pump.

In the second aspect, there is provided a use of the system according to the invention for storing and/or cultivating an organ or tissue model harvested from a donor or produced by other techniques, preferably with 3D bioprinting and/or 4D bioprinting and/or electrospinning.

Preferably, the system according to the invention is used for cultivating of a 3D bioprinted organ, wherein preferably the rotation mechanism is used at a stage of colonisation of a vascular system with endothelial cells.

Preferably, a state or development of a disease of an organ or a tissue model is evaluated during storing and/or cultivating.

Preferably, an effect of biologically active substances on the state or development of a disease of an organ or a tissue model is analysed during storing and/or cultivating.

Preferably, the efficacy of a pharmacological and/or gene therapy on the state of an organ or a tissue model is evaluated during storing and/or cultivating.

It is essential to maintain an organ or tissue model in normothermic conditions during cultivation, since it provides optimal metabolic conditions for an organ or a tissue model, corresponding to in vivo conditions, which allow for the condition of the organ to be evaluated.

Perfusion fluid for cultivating harvested organs contains without limitation a concentrate of red blood cells, which tends to sediment. The use of contactless means for mixing the perfusion fluid, preferably in the form of a mechanism that swings the perfusion fluid container or a container configuration that allows for inducing a vortex motion in the container, provides a homogeneous distribution of red blood cells in the perfusion fluid. Also, it allows for keeping the flow system sterile, and the mechanical damage to red blood cells, which occurs when using other types of mixers, is mitigated. The mixing of the perfusion fluid helps to heat the perfusion fluid evenly and constantly mix the components of the fluid.

The rotating mechanism for rotating the organ chamber is used without limitation when cultivating an organ produced by 3D bioprinting technology and is used at the stage of colonisation of the bioprinted vascular system with endothelial cells. Preferably, the rotating mechanism rotates the organ chamber by 180° every 30 minutes for at least two hours or at least four times by 90° every 15-60 minutes for at least two hours. The rotating mechanism enables colonization of the entire vascular system. Rotation is carried out in such a way that the cells have a chance to gravitationally fall on walls of channels and “stick” to them. The rotation may be performed at a smaller angle, e.g. 10 times by 36° for at least two hours, or even continuously—e.g. with a speed of 1 revolution per hour for at least two hours. The use of the rotating mechanism ensures homogeneous distribution of the colonised cells across the channel surface.

The system according to the invention may be used for cultivating and/or storing and/or treating (including regeneration) of the following:
organs harvested directly from deceased donors
organs harvested from living donors (e.g. family or crossover transplantations)
organs printed in 3D (and 4D) bioprinting technology (organs with flow system, including vascular system)
tissue models with flow system (including vascular system) printed in 3D (and 4D) bioprinting technology
organs and tissue models produced by electrospinning technology (organs with flow system, including vascular system)
organs and tissue models produced by a combination of the two methods (bioprinting and electrospinning)
organs harvested from breeding, laboratory and transgenic animals
organs subjected to genetic manipulation
organs produced under extrasystemic (laboratory) conditions using tissue and genetic engineering techniques.

Organs and tissue models cultivated in the system according to the invention may be used in medicine and for basic research as well as research and development work. Moreover, the system according to the invention may be used for cultivating tissue models with flow system (including vascular system), produced by means of 3D/4D bioprinting technology or other techniques that allow for producing models with a flow system (including electrospinning, which consists in obtaining nanofibres from melted polymers or solutions thereof using electric field), such as tissue models with cancerous foci formed, induced disease entities, tissue models suitable for gene therapy studies used for both research and personalised medicine.

The system may be a device used for storing and treating organs prior to transplantation, for testing the effects of biologically active substances, i.e. toxicity and efficacy, and for evaluating the severity of specific disease stages, as well as for evaluating the effectiveness of drug and gene therapies.

Advantageously, the system has a feature of precise dosage of medicaments, active substances and sampling for biological and chemical analyses. This makes it a multifunctional device which, in addition to storing/cultivating and treating organs, allows for conducting advanced research, preparing organs for transplantation and evaluating their functional state. Thus, it will contribute to the improvement of the effectiveness of transplantation procedures and will allow for conducting advanced research (both basic research and the pre-clinical phase).

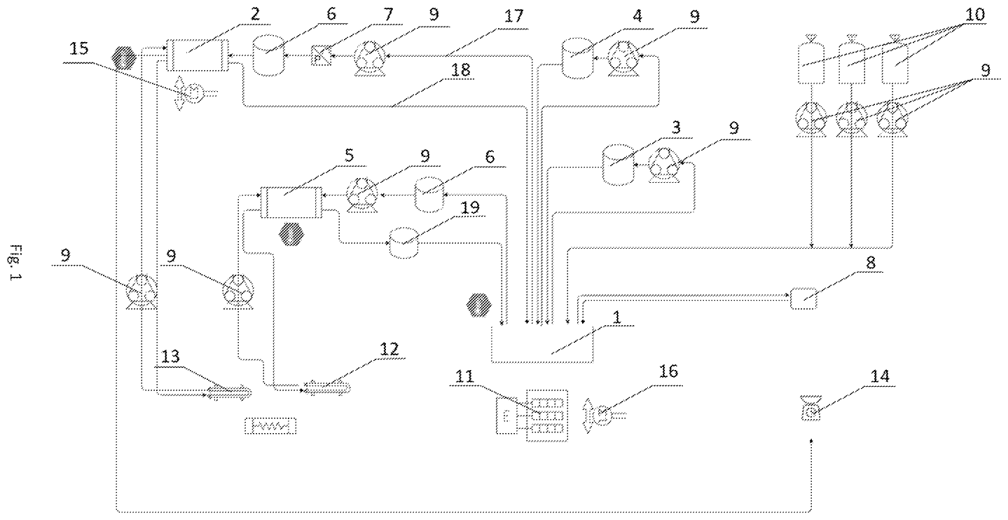

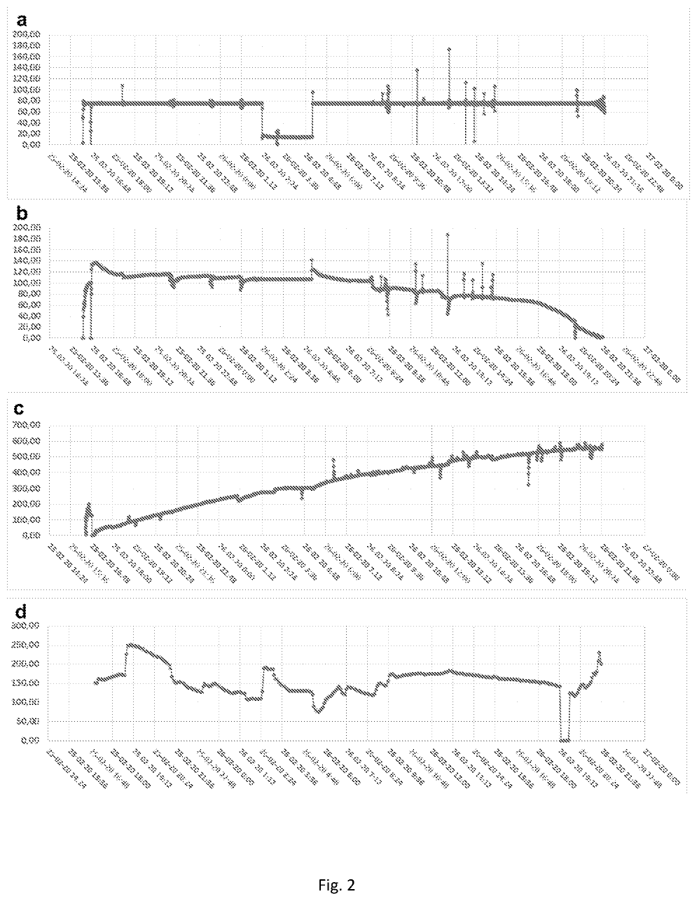

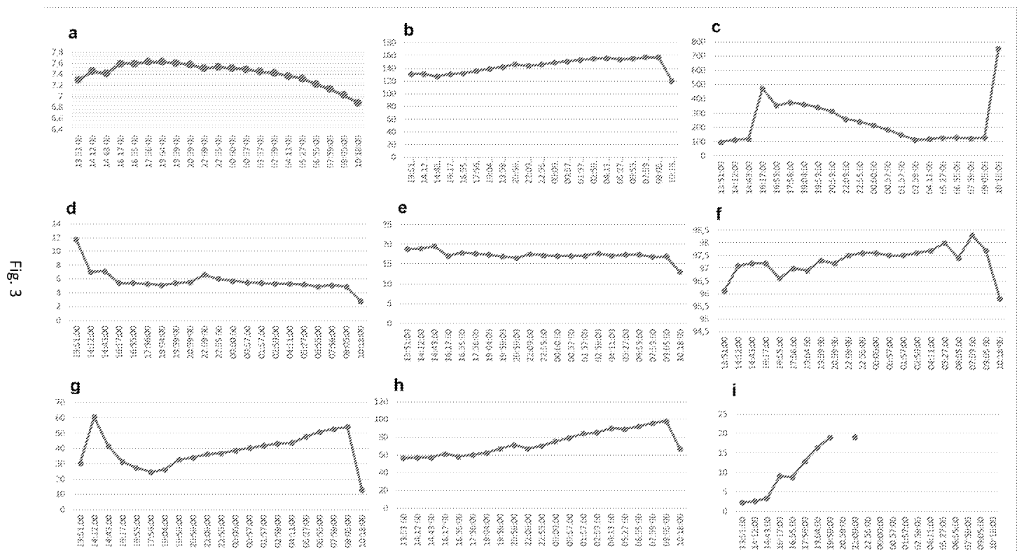

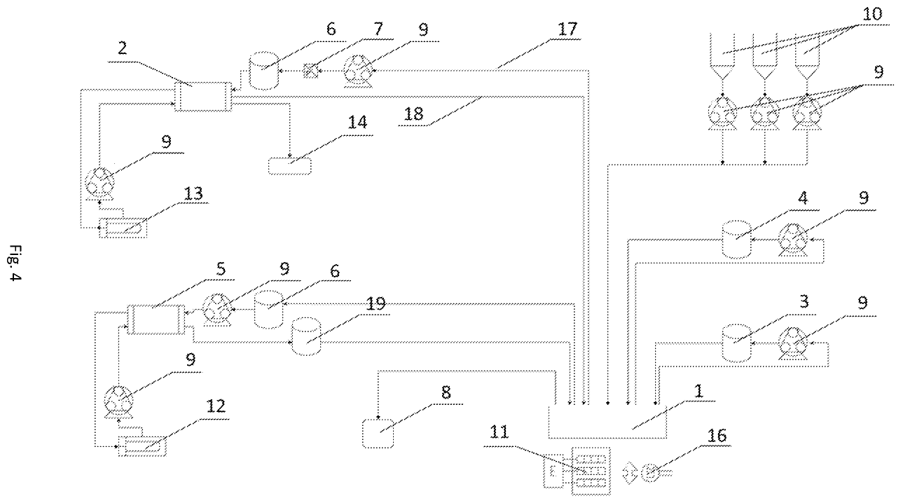

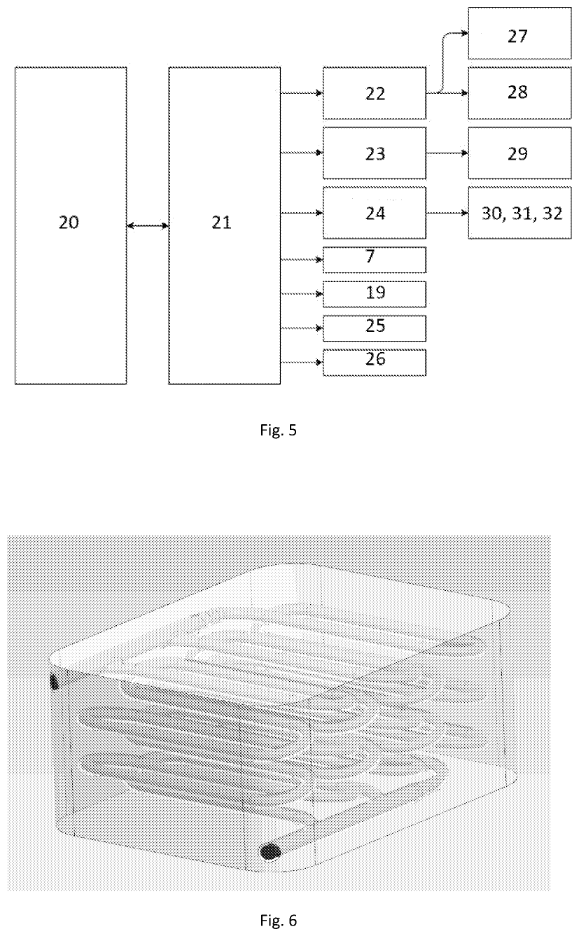

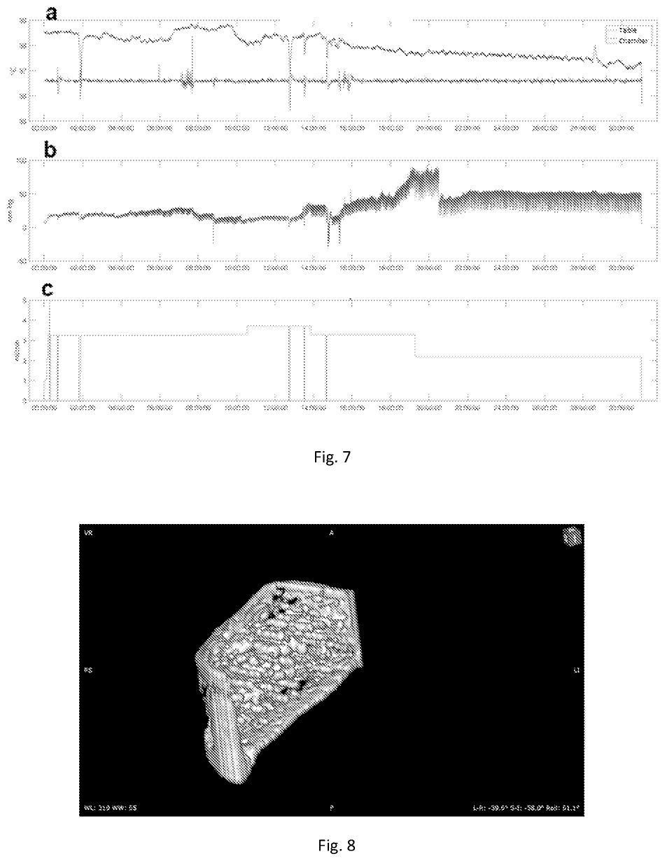

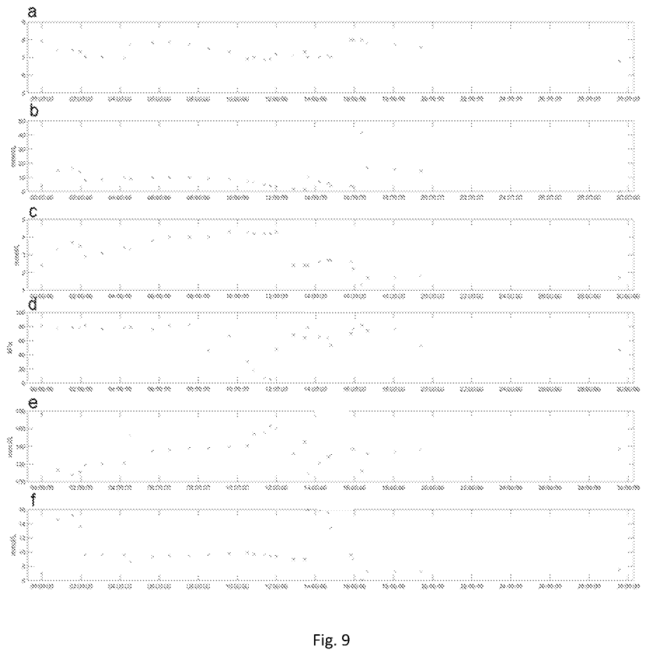

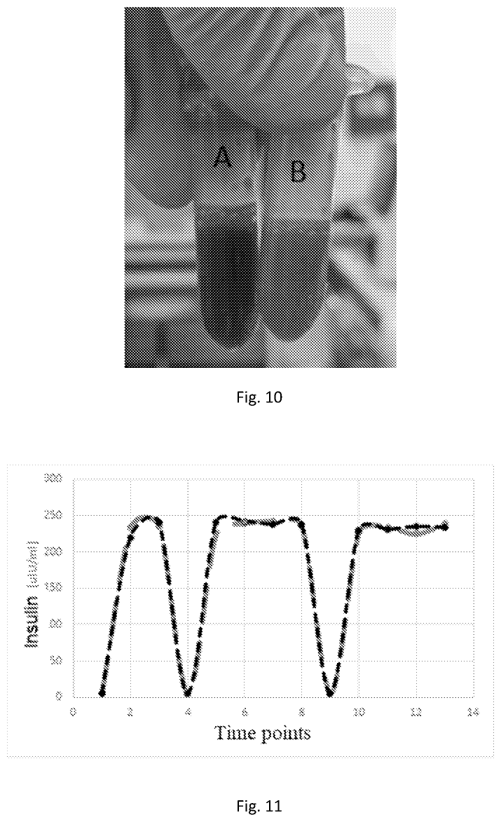

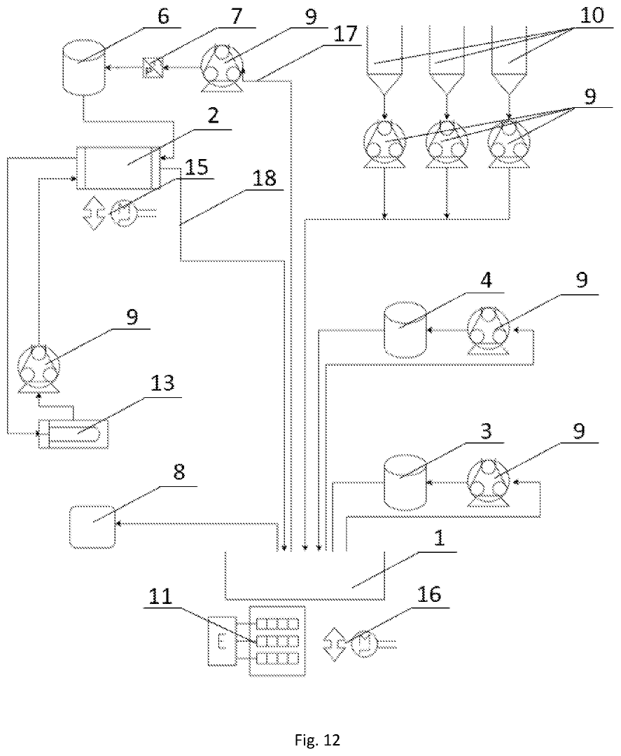

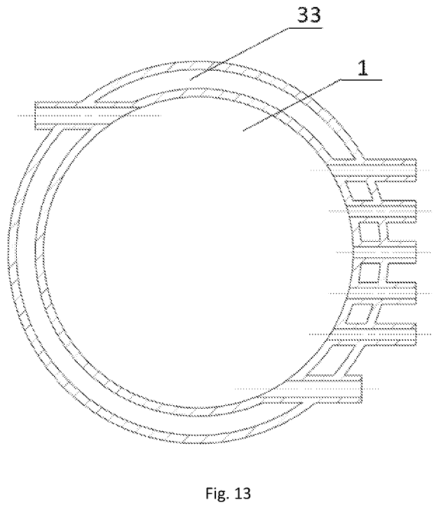

The invention is illustrated in embodiments in the drawing, where FIG. 1 shows a general diagram of the system, which allows for cultivating of both donor-harvested organs as well as organs or tissue models produced using other techniques; FIG. 2 shows a change in perfusion fluid parameters a) pressure [mm Hg], b) flow [ml/min.], c) loss of perfusion fluid [g] and d) glucose concentration [mg/ml] during a 2-day pig kidney cultivation in the system of Example 1, FIG. 3 shows the change in perfusion fluid parameters a) pH, b) Na⁺ ion concentration [mmol/l], c) glucose concentration [mg/dl], d) pCO₂ [kPA], e) K⁺ ion concentration [mmol/l], f) saturation (oxygenation) [%], g) pO₂ [kPA], h) Cl⁻ ion concentration [mmol/l] and i) lactate concentration [mmol/l] during cultivation of the pig kidney of Example 1, FIG. 4 shows the system used for cultivating the pig kidney from Example 1, FIG. 5 shows a block diagram of electronic system control means, FIG. 6 shows geometry of a bionic pancreas organ of Example 2, FIG. 7 shows monitoring of a) heating-cooling plate temperature (bottom line) and organ chamber temperature (top line) in ° C., b) arterial pressure RR [mm Hg], c) flow [ml/min.], FIG. 8 shows an image of the bionic pancreas obtained using a resonance method, FIG. 9 shows a change in perfusion fluid parameters a) pH, b) glucose concentration [mmol/l], c) lactate concentration [mmol/l], d) pO₂ [kPA], e) Na⁺ ion concentration [mmol/l], f) K⁺ ion concentration [mmol/l] during 30-hour cultivation of the bionic pancreas of Example 2, FIG. 10 shows a difference in oxygenation of the perfusion fluid after 30h: sample A—taken after leaving the chamber, in which the bionic organ was placed—deoxygenated fluid; sample B—taken before reaching the chamber—oxygenated fluid, FIG. 11 shows a difference in insulin concentration following stimulation with glucose solutions of variable concentration, where the grey line marks control—pancreatic islets subjected to glucose stimulation on inserts, and the black line marks pancreatic islets printed in the bionic pancreas, maintained in the system according to the invention, FIG. 12 shows the system used in Example 2 for cultivating the bionic pancreas, obtained in the 3D bioprinting process, FIG. 13 shows a cross-section of a perfusion fluid container according to Example 3.

### Example 1—Cultivating of a Pig Kidney

A pig kidney was used as a model when optimising the functions of the system according to the invention. The use of this organ was dictated by many pragmatic reasons:
easy accessibility and harvestability with minimised warm ischaemia time
vascularisation for easy connection
high metabolism of the organ, which allows for using the system's potential to the fullest
possibility to monitor organ function based on urine secretion.

Pig kidneys were harvested by interrupting technological cycle of meat production. After stunning an animal by current, it was exsanguinated, and directly after the process was over, the animal was transported to an operation room where the kidney was harvested (warm ischaemia time <10 min.). The harvest was completed when starting the arterial rinsing with UW ice solution (University of Wisconsin Solution). During the exsanguination of the animal, its blood was conserved in a sterile manner (heparinisation).

Composition of a perfusion fluid and conditions of organ cultivation in the system according to the invention:
A basic component of the perfusion fluid used during cultivating under normothermic conditions was low-potassium fluid for mechanical perfusion (Belzer MPS® UW Machine Perfusion Solution), enriched with red blood cell concentrate in a 7:3 ratio, which allowed for obtaining a hematocrit of approx. 0.15. Additionally, MPS liquid was supplemented with CaCl₂, aminoacid concentrate (Trimel N9-1070 EC), vitamins, sodium bicarbonate (to obtain a pH between 7.35 and 7.45) heparin, antibiotics (PenStrep), insulin and dexamethasone. The study compared average perfusion pressures of 50 mmHgh and 75 mmHg. Partial pressure of oxygen in an arterial branch of the system was approx. 50 mmHg with a flow through the oxygenator of approx. 3 L/min. The kidney has been cultivated for 48 hours and was then subjected to histopathological evaluation. Glucose concentration was maintained at 100-150 mg % by means of continuous infusion of glucose based on the perfusion fluid concentration. Losses resulting from diuresis were automatically replenished with Ringer fluid (in 1:1 ratio). The viability of the cultivated kidney was evaluated based on urine production, glucose consumption and blood saturation (oxygenation) upstream and downstream the organ. In case of a decrease in perfusion due to increased vascular resistance, the kidney was treated with an urapidil (Ebrantil) infusion (as needed). FIGS. 2 and 3 show changes in perfusion fluid parameters during cultivating of the pig kidney of Example 1.

The system used for cultivation of the pig kidney (FIG. 4) comprised the following:
a sealed organ chamber 2, equipped with means for adjusting temperature of the perfusion fluid placed in the chamber 2 during system operation. The chamber 2 contained a heating-cooling circuit 13 in a form of a heating jacket. The chamber 2 was equipped with ports allowing for connecting an organ, perfusion fluid inflow, perfusion fluid outflow, and a bubbletrap—an air bubble eliminator 6, designed to change the pressure or volume of gas contained in the chamber 2.
a perfusion fluid container 1 equipped with means for adjusting temperature of the perfusion fluid in a form of a heating-cooling plate 11, the temperature of which was adjusted by means of Peltier modules 27, wherein the perfusion fluid container 1 was connected to the chamber 2 by at least one first line 17, through which, during operation, the perfusion fluid could flow from the perfusion fluid container 1 to the organ chamber 2 and at least one second line 18 through which, during operation, the perfusion fluid could flow from the organ chamber 2 to the perfusion fluid container 1.
means for measuring and controlling of oxygenation of the perfusion fluid, comprising an oxygenation device: an oxygenator 5, oxygenation-saturation sensor 19 and a gas mixture cylinder assembly and a reducer, providing appropriate pressure and gas flow rate through the oxygenator 5. The oxygenator 5 also comprised a temperature sensor 32 of the oxygenator 5 and a heating-cooling circuit 12 in a form of a heating jacket of the oxygenator 5. The means for measuring and controlling oxygenation of the perfusion fluid are also referred to as the oxygenator 5 circuit.

The temperature of the perfusion fluid was adjusted by means of heating-cooling circuits 12 and 13 of the organ chamber 2 and the oxygenator 5, allowing the flow of heating or cooling fluid, heating-cooling circuit of the perfusion fluid container 1, temperature sensor 30 of the organ chamber 2, temperature sensor 31 of the perfusion fluid container 1, and temperature sensor 32 of the oxygenator 5, adjustment system and pumps 9, providing the flow of heating or cooling fluid, which was preferably distilled water, wherein the heating or cooling fluid did not come into contact with the organ or the perfusion fluid, but was only used to transfer heat from the heating device to the heating jacket of the organ chamber 2 and to the heating jacket of the oxygenator 5.
means 4 for measuring glucose concentration in the perfusion fluid and for dosing glucose into the perfusion fluid, comprising a sensor 25 based on an enzyme electrode, an electronic measuring system and a pump 9 for dosing glucose-containing supplementation fluid. An enzyme electrode was used, which is a disposable device specially designed for measurement in a flow system, having direct contact with the perfusion fluid. Glucose concentration data were displayed on a user's panel and based on this reading, the glucose-containing supplementation fluid was dosed by the system control means, either automatically or when needed by the operator.
means 3 for measuring pH of the perfusion fluid and for dosing substance for adjusting pH of the perfusion fluid, comprising a pH electrode, an electronic measuring system and a pump for dosing substance for adjusting pH
means for contactless mixing of the perfusion fluid provided in a form of a swinging mechanism 16 of the perfusion fluid container 1. The swinging mechanism 16 caused a swinging movement off perfusion fluid container 1.
means for air removal from perfusion fluid comprising two perfusion fluid bubbletraps—air bubble eliminators 6. One bubbletrap was placed in the oxygenator 5 circuit, and the other bubbletrap (described above) was placed directly before the organ chamber 2.
means for measuring and controlling of flow parameters of the perfusion fluid, including pressure of the perfusion fluid, which during operation of the system flows from the perfusion fluid container 1 into the organ chamber 2. The means for measuring and controlling of flow parameters of the perfusion fluid comprised an electro-mechanical membrane pressure sensor 7, an electronic control system and a peristaltic pump 9. Pressure measurement was conducted in a contactless manner. The perfusion fluid was separated from the sensor 7 by a flexible membrane. The perfusion pressure was set by perfusion operator on the user panel using the system control means. The system control means automatically selected a flow rate so as to achieve the set pressure. The maximum permissible flow rate was also preset on the user panel.
means 14 for measuring of loss and replenishment of the perfusion fluid, comprising a weight based on a tensometric sensor, an electronic measuring system and a dosing pump 9.

An amount of added fluid was displayed on the operator's panel and, depending on the settings, it could be filled up automatically or manually by the operator of the device using the system controls means.
system control means, configured and programmed to obtain measurement values for at least one parameter selected from: temperature of the perfusion fluid in the organ chamber 2, temperature of the perfusion fluid in the perfusion fluid container 1, concentration of glucose in the perfusion fluid, pH of the perfusion fluid, parameters of the perfusion fluid flow, including pressure of the perfusion fluid and/or output of the perfusion fluid, expressed as a unit of volume per unit of time, concentration of lactates in the perfusion fluid, concentration of sodium ions in the perfusion fluid, concentration of chlorine ions in the perfusion fluid and concentration of potassium ions in the perfusion fluid, oxygenation of the perfusion fluid and automatic and/or manual adjustment based on predetermined criteria of at least one parameter selected from: temperature of the perfusion fluid in the organ chamber, temperature of the perfusion fluid in the perfusion fluid container, concentration of glucose in the perfusion fluid, pH of the perfusion fluid, parameters of the perfusion fluid flow, including pressure of the perfusion fluid and/or output of the perfusion fluid, lactate concentration in the perfusion fluid, sodium ion concentration in the perfusion fluid, chlorine ion concentration in the perfusion fluid and potassium ion concentration in the perfusion fluid, oxygenation of the perfusion fluid.

A main controller of the system control means consisted of two parts: the first one based on a microprocessor 21, which ensured real-time operation by controlling all the peripherals of the device. The second processor 20 was responsible for the graphical interface (GUI), enabling observation and input of parameters.

A TEC controller 23 was designed to control the Peltier modules 27 so that they maintain the perfusion fluid temperature between 0 and 37 degrees C., allowing for handling the organ under both normothermic and hypothermic conditions. Fans 28 were used to control radiator temperature so that the temperature difference between the hot and cold side of the Peltier module is not more than 20° C.

The controller 22 recorded the following temperature measurements: measurement of the Peltier modules 27, measurement of the perfusion fluid heating-cooling plate 11, measurement in the water jacket of the organ chamber 2, measurement in the perfusion fluid container 1.

Several peristaltic pumps 9 were used, the first of which was used for the main perfusion fluid circulation and was controlled to maintain a constant fluid pressure controlled by a tensometric pressure sensor 7. Three more pumps 9 were used to administer medicinal substances. It was possible to administer medicaments at a specific rate in ml/min.

The control panel of the system according to the invention could be accessed from a level of a web browser, so that it could be opened with any device such as a mobile phone, tablet or laptop, provided it had access to the Internet.

The control panel allowed for setting such parameters as: temperature of the heating-cooling plate 11, parameters of the PID controller for temperature control, switching on and off pressure control, preset perfusion fluid pressure [mmHg], minimum and maximum flow (output) of perfusion fluid, presetting speed [ml/min.] of each stepper motor 29, switching on the perfusion fluid mixing mechanism, setting the perfusion fluid mixing speed. The control panel also allowed for monitoring the measured parameters such as pH of the perfusion fluid, glucose concentration in the perfusion fluid, temperature of the heating-cooling plate 11. FIG. 5 is a block diagram of the electronic system control means.

### Example 2—Cultivating of an Organ Obtained with a Bioprinting Technology

A bionic pancreas organ was used to conduct an experiment implemented using 3D bioprinting. The organ comprised a scaffolding, containing pancreatic islets, made of a base bioink and a system of ducts, made of a vascular bioink (FIG. 6). The duct system consisted of a primary vessel, branching into three spirally arranged secondary vessels and merging into one discharging vessel. Description of geometrical parameters of the organ:
External dimensions of the whole model were 32×40×17.5 mm
The length of a single secondary vessel was 340 mm
Total length of secondary vessels was 1020 mm
Diameter of primary vessels was 1.5 mm
Diameter of secondary vessels was 1 mm
Scaffolding volume for pancreatic islets was 20.5 ml
Volume of the ducts was approx. 0.9 ml

Cultivation process was conducted in controlled temperature conditions: organ chamber 2 temperature in a range between 37 and 39° C.; temperature of heating-cooling plate 11 for heating liquids in a range between 36.5 and 37° C. During the whole experiment, flow and pressure level were also monitored (FIG. 7).

A manner of connecting the bionic organ to the organ chamber 2 and monitoring of parameters (pressure, flow, temperature) did not affect structure and function of the vascular system printed in the bionic organ. This was confirmed by a resonance method (FIG. 8).

During the incubation of the bionic organ in the organ chamber 2, the following parameters of the perfusion fluid were monitored in real time: oxygenation, pH, glucose concentration, lactate concentration, sodium ions, potassium ions (FIG. 9).

Control of parameters allowed for monitoring of composition of the perfusion fluid and, if needed, for improving its quality, which made it possible to maintain the bionic organ in optimum cultivation/incubation conditions. Moreover, the perfusion fluid used in the system was supplemented with red blood cells, which were a biological oxygen carrier. During the experiment (after 30h), perfusion fluid samples were taken, showing a clear difference in perfusion fluid oxygenation (FIG. 10). Sample A—taken after leaving the chamber 2, in which the bionic organ was placed—deoxygenated fluid; sample B—taken before reaching the chamber 2—oxygenated fluid.

Also, tests were carried out on the bionic organ to evaluate its functionality, which, apart from the control of the basic parameters described above, also involved taking samples at set time points to assess insulin concentration after stimulation with glucose solutions of variable concentration (FIG. 11). The bionic organ results did not differ between isolated pancreatic islets which were printed as the bionic organ and islets that were not subject to the 3D bioprinting process.

A system used for cultivation of the bionic organ (FIG. 12) comprised the following:
a sealed moveable organ chamber 2, equipped with means for adjusting temperature of the perfusion fluid placed in the chamber 2 during the operation of the device. Chamber 2 contained a heating-cooling circuit 13 in a form of a heating jacket, a rotating mechanism 15 of the chamber 2 for rotating organ chamber 2 and a chamber 2 rotation drive. The chamber 2 was equipped with ports allowing for connecting an organ, perfusion fluid inflow, perfusion fluid outflow, and an air bubble eliminator 6, designed to change the pressure or volume of gas contained in the chamber 2.
a perfusion fluid container 1 equipped with means for adjusting the temperature of the perfusion fluid in a form of a heating-cooling plate 11, the temperature of which was adjusted by means of Peltier modules 27, wherein the perfusion fluid container 1 was connected to the chamber 2 by at least one first line 17, through which, during operation, the perfusion fluid could flow from the perfusion fluid container 1 to the organ chamber 2 and at least one second line 18 through which, during operation, the perfusion fluid could flow from the organ chamber 2 to the perfusion fluid container 1.

Temperature of the perfusion fluid was adjusted by means of heating-cooling circuits of the organ chamber 2 and the perfusion fluid container 1, a temperature sensor 30 of the organ chamber 2, a temperature sensor 31 of the perfusion fluid container 1, a control system and pumps 9 providing the flow of heating or cooling fluid. The organ chamber 2 had a heating-cooling circuit 13, allowing for flow of heating or cooling fluid.
means 4 for measuring glucose concentration in the perfusion fluid and for dosing glucose into the perfusion fluid, comprising a sensor 25 based on an enzyme electrode, an electronic measuring system and a pump 9 for dosing a glucose-containing fluid. An enzyme electrode was used, which is a disposable device specially designed for measurement in a flow system, having direct contact with the perfusion fluid. Glucose concentration data were displayed on a user's panel and based on this reading, supplementation fluid was dosed by the system control means, either automatically or when needed by the operator.
means 3 for measuring pH of the perfusion fluid and for dosing substance for adjusting pH of the perfusion fluid comprising a pH electrode, an electronic measuring system and a pump 9 for dosing substance for adjusting pH.
means for contactless mixing of the perfusion fluid provided in a form of a swinging mechanism 16 of the perfusion fluid container 1. The swinging mechanism 16 caused a swinging movement off perfusion fluid container 1.
means for air removal from the perfusion fluid comprising a perfusion fluid bubbletrap—air bubble eliminator 6 located directly before the organ chamber 2.
means for measuring and controlling of flow parameters of the perfusion fluid, including pressure of the perfusion fluid, which during operation of the system flows from the perfusion fluid container 1 into the organ chamber 2. The means for measuring and controlling of flow parameters of the perfusion fluid comprised an electro-mechanical membrane pressure sensor 7, an electronic control system and a peristaltic pump 9. Pressure measurement was conducted in a contactless manner. The perfusion fluid was separated from the sensor 7 by a flexible membrane. The perfusion pressure was set by perfusion operator on the user panel using the system control means. System control means automatically selected a flow rate so as to achieve the set pressure. The maximum permissible flow rate was also preset on the user panel.
system control system matched those in Example 1. The system control means further allowed for controlling rotation of the organ chamber 2.

### Example 3

For cultivation of the pig kidney of Example 1, a system was used that corresponded to that of Example 1, in which the perfusion fluid container 1 was not equipped with a swinging mechanism 16 and heating using Peltier modules 27. The container 1 was cylindrical in shape with ports located in its lower section (FIG. 13). Contactless mixing of the perfusion fluid was achieved by introducing a vortex motion in container 1, by placing the inlet and outlet of perfusion fluid of an oxygenator 5 circuit on opposite sides of the perfusion fluid container 1, tangentially to its cylindrical walls. Fluid flow output at the inlet and the outlet of the oxygenator 5 circuit was set between 0 and 2 L/min. Heating or cooling of the perfusion fluid was conducted by using a water jacket 33. Perfusion fluid container 1 was produced in 3D printing technology.

List of references in drawings: 1-perfusion fluid container; 2-organ chamber; 3 means for measuring pH; 4-means for measuring glucose concentration; 5-oxygenator; 6-air bubble eliminator; 7-pressure sensor of perfusion fluid; 8-means for taking samples of perfusion fluid; 9-peristaltic pump; 10-container for medicaments and/or nutrients and/or perfusion fluid components; 11-heating-cooling plate; 12-heating-cooling circuit of an oxygenator; 13-heating-cooling circuit of an organ chamber; 14-means for measuring of loss of perfusion fluid; 15-rotating mechanism for rotating the organ chamber; 16-swinging mechanism of perfusion fluid container; 17-first line; 18-second line; 19-oxygenation/saturation sensor; 20-GUI processor; 21-real-time microprocessor; 22-TEC controller; 23-stepper motor controllers; 24-temperature measurement system; 25-glucose sensor; 26-liquid level sensor; 27-Peltier modules; 28-fans; 29-stepper motors; 30-temperature sensor of organ chamber; 31-temperature sensor of perfusion fluid container; 32-temperature sensor of oxygenator, 33-water jacket of perfusion fluid container of Example 3.

1. A system for storing or cultivating of an organ or a tissue model, preferably harvested from a donor, preferably selected from pancreas, liver, kidney, lung, heart, small intestine, large intestine, thyroid, skin, brain, or produced using other techniques, preferably using 3D bioprinting and/or 4D bioprinting and/or electrospinning technologies, preferably in a form of organs and/or tissue models printed with flow or vascular systems, comprising:
a chamber (2) for an organ or a tissue model equipped with means for measuring and adjusting temperature of perfusion fluid placed in the chamber (2) during operation of the system, wherein said means for measuring and adjusting temperature of the perfusion fluid preferably comprise a water jacket
a perfusion fluid container (1), equipped with means for measuring and adjusting temperature of the perfusion fluid in the container (1) during the operation of the system, preferably comprising a heating-cooling plate (11), wherein the perfusion fluid container (1) is connected to the organ chamber (2) by at least one first line (17) through which the perfusion fluid can flow from the perfusion fluid container (1) to the organ chamber (2) during operation, and at least one second line (18) through which the perfusion fluid can flow from the organ chamber (2) to the perfusion fluid container (1) during operation
means (4) for measuring glucose concentration in the perfusion fluid and for dosing glucose into the perfusion fluid
means (3) for measuring pH of the perfusion fluid and for dosing substance for adjusting the pH of the perfusion fluid
means for contactless mixing of the perfusion fluid
means for measuring and controlling of flow parameters of the perfusion fluid, including perfusion fluid pressure and/or perfusion fluid output, expressed as a unit of volume per unit of time
optionally, means for measuring and controlling one or more parameters, selected from: lactate concentration in the perfusion fluid, concentration of sodium ions in the perfusion fluid, concentration of chlorine ions in the perfusion fluid, concentration of potassium ions in the perfusion fluid, oxygenation level of the perfusion fluid, wherein the system is configured to circulate said perfusion fluid during operation of the system between the organ chamber (2) and the perfusion fluid container (1).

2. The system for storing or cultivating of an organ or tissue model according to claim 1, which comprises system control means, configured and programmed to obtain measurement values for at least one parameter selected from: temperature of the perfusion fluid in the organ chamber (2), temperature of the perfusion fluid in the perfusion fluid container (1), concentration of glucose in the perfusion fluid, pH of the perfusion fluid, parameters of the perfusion fluid flow, including pressure of the perfusion fluid and/or output of the perfusion fluid, expressed as a unit of volume per unit of time, concentration of lactates in the perfusion fluid, concentration of sodium ions in the perfusion fluid, concentration of chlorine ions in the perfusion fluid and concentration of potassium ions in the perfusion fluid, oxygenation level of the perfusion fluid, wherein the system control means are further configured and programmed for automatic and/or manual adjustment based on predetermined criteria of the at least one parameter.

3. The system for storing or cultivating of an organ or tissue model according to claim 2, which further comprises means for measuring and controlling the oxygenation of perfusion fluid and/or means for air removal from the perfusion fluid, wherein the system control means are preferably configured and programmed to obtain perfusion fluid oxygenation measurement values and automatic and/or manual adjustment of perfusion fluid oxygenation based on predetermined criteria.

4. The system for storing or cultivating of an organ or tissue model according to claim 1 or 2 or 3, which further comprises means (14) for measuring of loss and replenishment of the perfusion fluid, preferably based on a reading of weight of the perfusion fluid removed from the system, wherein the system control means are preferably configured and programmed for automatic and/or manual replenishment of the perfusion fluid based on predetermined criteria.

5. The system for storing or cultivating of an organ or tissue model according to claim 1 or 2 or 3 or 4, wherein the means for contactless mixing of the perfusion fluid are provided as a swinging mechanism (16) for swinging the perfusion fluid container (1), wherein the system control means are preferably configured and programmed to automatically and/or manually adjust the swinging of the perfusion fluid container (1).

6. The system for storing or cultivating of an organ or tissue model according to claim 3 or 4, wherein the oxygenation of the perfusion fluid is performed with an oxygenator (5), which is peripherally connected to the perfusion fluid container (1) to form an oxygenator (5) circuit, and the means for contactless mixing the perfusion fluid are provided in a form of a cylindrical design of the perfusion fluid container (1), in which, during operation of the system, the perfusion fluid is mixed by placing inlet and outlet of the oxygenator circuit (5) in the perfusion fluid container (1) on opposite sides of the perfusion fluid container (1), tangentially to cylindrical walls of the perfusion fluid container (1).

7. The system for storing or cultivating of an organ or tissue model according to any one of the claims 1 to 6, comprising means (8) for taking samples of the perfusion fluid.

8. The system for storing or cultivating of an organ or tissue model according to any one of the claims 1 to 7, wherein the organ chamber (2) comprises a rotating mechanism (14) which allows the organ chamber (2) to rotate during operation of the system, wherein the system control means are preferably configured and programmed to automatically and/or manually adjust the rotation of the organ chamber (2), wherein the system control means are preferably configured and programmed to rotate the organ chamber (2) by 180° every 30 minutes for at least two hours or at least four times by 90° every 15-60 minutes for at least two hours

9. The system for storing or cultivating of an organ or tissue model according to any one of the claims 1 to 8, wherein the organ chamber (2) and/or the perfusion fluid container (1) and/or the means for measuring and controlling the oxygenation of the perfusion fluid has/have means for adjusting temperature in the range from 0 to 37° C.

10. The system for storing or cultivating of an organ or tissue model according to any one of the claims 1 to 9, which further comprises additional means for dosing medicaments and/or nutrients and/or perfusion fluid components, wherein the system control means are preferably configured and programmed for automatic and/or manual dosing medicaments and/or nutrients and/or perfusion fluid components based on predetermined criteria.

11. The system for storing or cultivating of an organ or tissue model according to any one of the claims 3 to 10, wherein air is removed from the perfusion fluid by means of an air bubble eliminator (6) mounted on the first line (17) between the perfusion fluid container (1) and the organ chamber (2), and the movement of the perfusion fluid between the perfusion fluid container (1) and the organ chamber (2) and the other way round during operation of the system is performed by means of at least one pump (9), preferably a peristaltic pump.

12. Use of the system as defined in any one of the claims 1 to 11 for storing and/or cultivating of an organ or tissue model harvested from a donor or produced by other techniques, preferably with 3D bioprinting and/or 4D bioprinting and/or electrospinning.

13. The use according to claim 12, wherein the system of claim 8 is used for cultivating of a 3D bioprinted organ, wherein preferably the rotation mechanism (14) is used at a stage of colonisation of a vascular system with endothelial cells.

14. The use according to claim 12, wherein a state or development of a disease of an organ or a tissue model is evaluated during storing and/or cultivating.

15. The use according to claim 14, wherein an effect of biologically active substances on the state or development of a disease of an organ or a tissue model is analysed during storing and/or cultivating.

16. The use according to claim 14, wherein the efficacy of a pharmacological and/or gene therapy on the state of an organ or a tissue model is evaluated during storing and/or cultivating.
